(19) United States
(12) Patent Application Publication
(10) Pub. No.: US 2024/0153602 A1
(43) Pub. Date: May 9, 2024

(54) BLOCKCHAIN-BASED PLATFORM FOR HEALTH RECORD EXCHANGE

(71) Applicant: Blue Cross and Blue Shield of Massachusetts, Inc., Boston, MA (US)

(72) Inventors: Vijay Kukreja, Braintree, MA (US); Scott Haigh, Canton, MA (US)

(21) Appl. No.: 18/413,789

(22) Filed: Jan. 16, 2024

Related U.S. Application Data
(63) Continuation of application No. PCT/US2022/037127, filed on Jul. 14, 2022.
(60) Provisional application No. 63/221,814, filed on Jul. 14, 2021.

(51) Int. Cl. G16H 10/60; G06F 21/60

(52) U.S. Cl. CPC G16H 10/60; G06F 21/602

(57) ABSTRACT

Systems, methods, and computer program products are provided for blockchain-based healthcare record transfer and sharing that is secure and auditable. In various embodiments, a method is provided where a request is received from a user for one or more healthcare records. A selection of one or more secure providers is received from the user. The one or more healthcare records are retrieved from a healthcare record database. The one or more healthcare records are combined into a healthcare record package. A hash is generated based on the healthcare record package. The healthcare record package is transmitted to the one or more secure providers. The user, the one or more secure providers, and the hash is recorded in a blockchain ledger (while not recording any data from within the healthcare records).

### CROSS-REFERENCE TO RELATED APPLICATIONS

This application is a continuation of International Application No. PCT/US2022/037127, filed Jul. 14, 2022, which claims the benefit of U.S. Provisional Application No. 63/221,814, filed Jul. 14, 2021, each of which is hereby incorporated by reference in its entirety.

### BACKGROUND

Embodiments of the present disclosure relate to blockchain-based healthcare record transfer and sharing.

### BRIEF SUMMARY

Systems, methods, and computer program products are provided for blockchain-based healthcare record transfer and sharing. In various embodiments, a request is received from a user for one or more healthcare records. A selection of one or more secure providers is received from the user. The one or more healthcare records are retrieved from a healthcare record database. The one or more healthcare records are combined into a healthcare record package. A hash is generated based on the healthcare record package. The healthcare record package is transmitted to the one or more secure providers. The user, the one or more secure providers, and the hash is recorded at a block in a blockchain ledger.

### BRIEF DESCRIPTION OF THE SEVERAL VIEWS OF THE DRAWINGS

FIG. 1 illustrates a process of healthcare record transfer and sharing between consumers, different providers, and facilities according to embodiments of the present disclosure.

FIGS. 2A-2B illustrate a process of healthcare record transfer and sharing between consumers, different providers, and facilities using a blockchain-based approach according to embodiments of the present disclosure.

FIG. 3A illustrates a flow diagram of a process for recording a transaction on a blockchain ledger according to embodiments of the present disclosure. FIG. 3B illustrates a flow diagram of a process for storing a transaction ID and hash in an HSP system according to embodiments of the present disclosure.

FIGS. 4A-4G illustrate various snapshots of a mobile phone application according to embodiments of the present disclosure.

FIG. 5 depicts an exemplary computing node according to embodiments of the present invention.

### DETAILED DESCRIPTION

The present disclosure relates to systems, methods, and computer program products for enabling smart and secure healthcare interactions—specifically, sharing and transfers of healthcare records. With new mandates for transparency and data sharing, it is important that the consumer has the ability to access, select, and transfer their healthcare related information when needed, including only the relevant data for the specified purpose, and that the transfer is recorded and delivered securely to the selected recipients. The present disclosure provides for control over these healthcare record transactions such that the consumer may be certain that these transactions are secure, monitored, and can be readily audited if needed.

A Health Data Share & Protect (HSP) network is a business-to-business (b2b) and/or business-to-consumer (b2c), member-controlled, interoperability platform that allows members to digitally send and receive health records within the network. Moreover, the platform may allow for safe and fast app-to-app interoperable transactions between healthcare institutions (e.g., Payers, Providers, Pharmacies, and others within the HSP network). In various embodiments, Payers, Providers, Pharmacies and/or Employers are Business Entities/Organizations that may subscribe to be part of the HSP network. In various embodiments, these Business Entities/Organizations are incentivized to become verified members of the HSP network and to help enable frictionless healthcare information exchange leveraging the HSP network. In various embodiments, Payers may be able to share and receive various kinds of health records like insurance policies, plans, coverage, claims, and/or provider directories. In various embodiments, Providers and Pharmacies may be able to share and receive specific records related to Conditions and Medications, such as, for example, Specialty Drugs, counseling records, etc. In various embodiments, Employers may be able to share and receive Plan and Benefits records and Employee Verification records. In various embodiments, Payers, Providers, Pharmacies and/or Employers may be able to send and/or receive various kinds of healthcare information via the HSP network.

In various embodiments, the HSP network makes healthcare record transactions easy, safe, and fast by validating and certifying various partners on a regular basis—inside and outside a private blockchain network. In various embodiments, healthcare records may be sent via the HSP platform and network using an email address. In various embodiments, healthcare records may be sent via the HSP platform and network using a mobile phone number. In various embodiments, the HSP application may leverage Fast Healthcare Interoperability Resources (FHIR) and/or other standard protocols from within the HSP application. In various embodiments, the HSP application may be available on mobile devices and/or web devices.

In various embodiments, an email address and/or a mobile phone number may provide a unique account ID, enabling the user to authenticate with the HSP. In various embodiments, the phone number may be associated with a mobile device. In various embodiments, the email address may be associated with an email account of record. In various embodiments, any suitable known techniques for authentication may be used to 1) enable registration and 2) to secure transactions. In various embodiments, authentication may include sending a unique time-bound token to the user's selected verification channel (e.g., an e-mail or mobile text message). In various embodiments, any suitable 2-factor authentication (2FA) procedure may be used. For example, an occasional PIN or other 2FA validation may be performed at predetermined times (e.g., weekly) to ensure the channel security has not changed. In various embodiments, the email and/or mobile number may be used depending on the user's preference to deliver confirmations of transactions and notifications regarding the user's healthcare records.

In various embodiments, a comprehensive transactional log of the details of the healthcare record exchange may be maintained on the HSP blockchain ledger for audit, certification and tracking purposes. In various embodiments, the log stored on the blockchain does not include the healthcare record (i.e., the data) itself. In various embodiments, the log include a hash of the data contained within the healthcare record. In various embodiments, the transaction details may be stored on the blockchain. For example, the data field list of a healthcare record sent to a specific provider may be stored in a block on the blockchain including the time, date, and recipient of the healthcare record. In various embodiments, the healthcare record is maintained in the healthcare record database (e.g., EHR) along with a record of any changes or updates. In various embodiments, the combination of the health record audit log and the on-chain transaction may be used to track what healthcare data was sent to the target provider for a given field. In various embodiments, the actual data stored within the healthcare record may be shared or transferred via a FHIR protocol from existing interoperability frameworks in parallel to the blockchain ledger management of the HSP.

In various embodiments, a comprehensive audit log of the any health record updates may be maintained in the HSP system. In various embodiments, the actual healthcare record and any changes (e.g., changes in data, date and time of changes, etc.) may be stored as the healthcare record evolves in the HSP health database.

In various embodiments, a machine learning algorithm (e.g., neural network) may monitor and track for any abnormal transactions and raise alerts to the appropriate administrators. In various embodiments, the machine learning algorithm may detect multiple transaction requests to select portions or send or complete health records to non-HSP providers. In various embodiments, the machine learning algorithm may detect patterns of changes to devices, emails, phone numbers, etc. that are unusual for a given user. In various embodiments, the machine learning algorithm may detect abnormal patterns in requesting records. In various embodiments, the machine learning algorithm may detect abnormal updating of records by providers, by an EHR, or by consumers.

In various embodiments, the machine learning algorithm may detect an abnormal request based on location data of the requesting device (e.g., if location services are activated).

In various embodiments, the HSP network may be used to transfer or share, for example, 1.) fitness related health information to gym or fitness coach—a fitness provider, may provide nutritional information, or health related information (diabetes, heart, exercise) information; 2.) allergy information sharing with a pharmacy provider; 3.) general health information sharing with specific providers; 4.) vision related information for optometric providers; 5.) other similar condition specific health data sharing with specialty providers.

In various embodiments, the HSP system may include a private secure health record datastore within the HSP internal health record database. In various embodiments, the HSP system may include a parallel blockchain secured transaction within the HSP internal health record database.

In various embodiments, the datastore and its associated secure servers, applications and API integrations may provide health record data transfer services. In various embodiments, the blockchain system may record a hash of all transactions, such that all healthcare record sharing via the HSP platform may be tracked and auditable. In various embodiments, a Hardware security module (HSM) may be included as part of the system architecture to provide (non-software based) encryption capability and key management. In various embodiments, the HSM may be used for health record, profile, and/or other sensitive data at rest and in-transit data encryption.

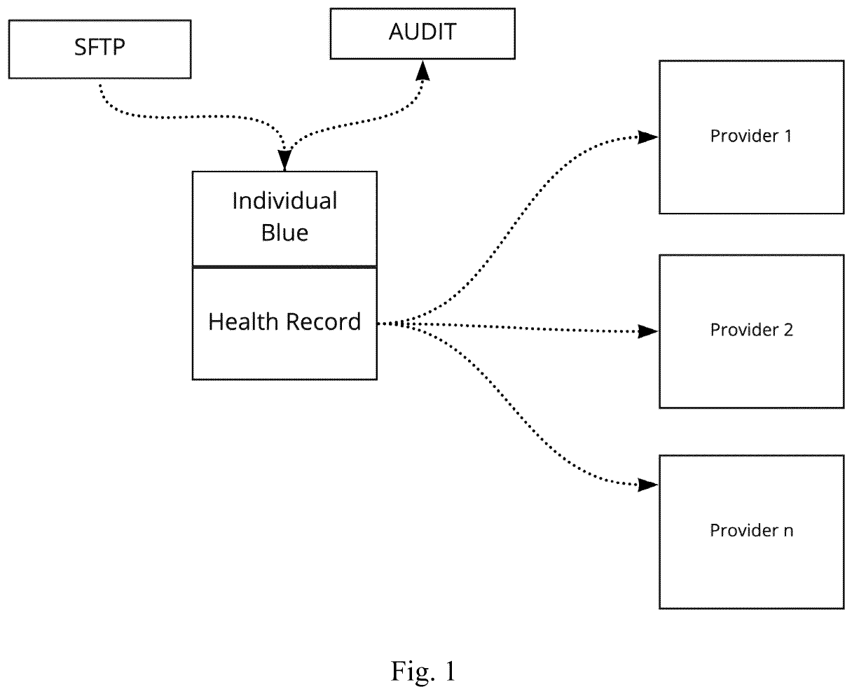

FIG. 1 illustrates a process of healthcare record transfer and sharing between consumers, different providers, and facilities. In particular, FIG. 1 represents a traditional approach to health record transfer and/or sharing between consumers, different providers, and facilities that is non-uniform (e.g., utilizing many different sharing approaches, different standards, custom interfaces, etc.) With the non-uniformity of the health care record sharing comes potential security risks in addition to compliance issues. Many exchanges have point-to-point communication and little or no transactional audit capability of healthcare record transfers and/or record sharing. In particular, a secure file transfer protocol may be initiated directly with a healthcare record server (such as a Blue Cross Blue Shield healthcare record server, Individual Blue) to perform an audit. To perform an audit of healthcare records, the records themselves are required to be downloaded via the secure transfer protocol. This process requires significant processing and bandwidth resources and is not suitable for the scaling of auditing functions to providing auditing tools internally, to customers, and/or to healthcare providers. Moreover, one or more providers may request healthcare records from a healthcare record server. However, the requests for medical records are not recorded and, thus, auditing this information—specifically, what information was sent, when it was sent, and to whom it was sent—may prove to be difficult if not impossible.

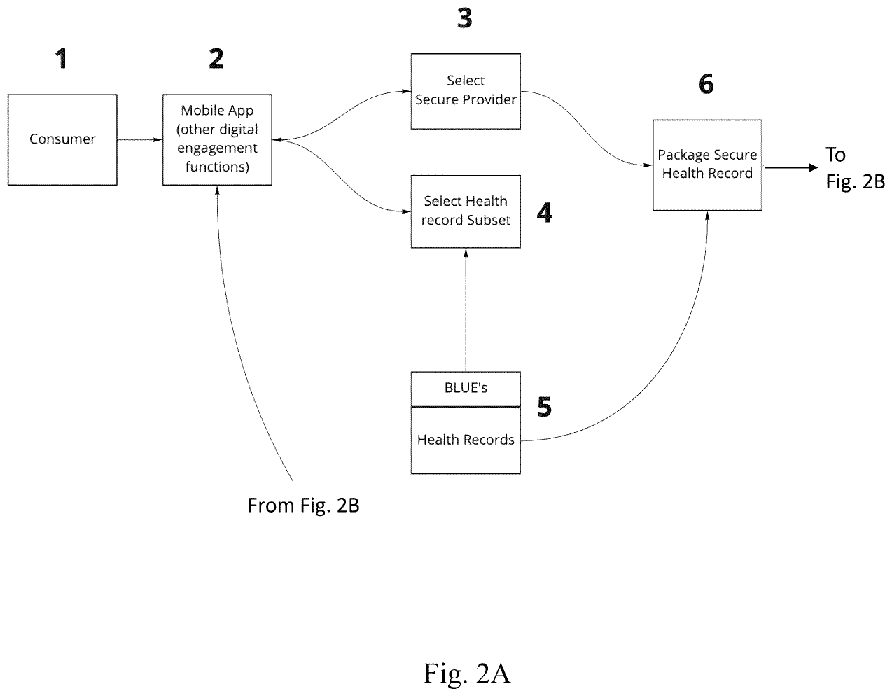

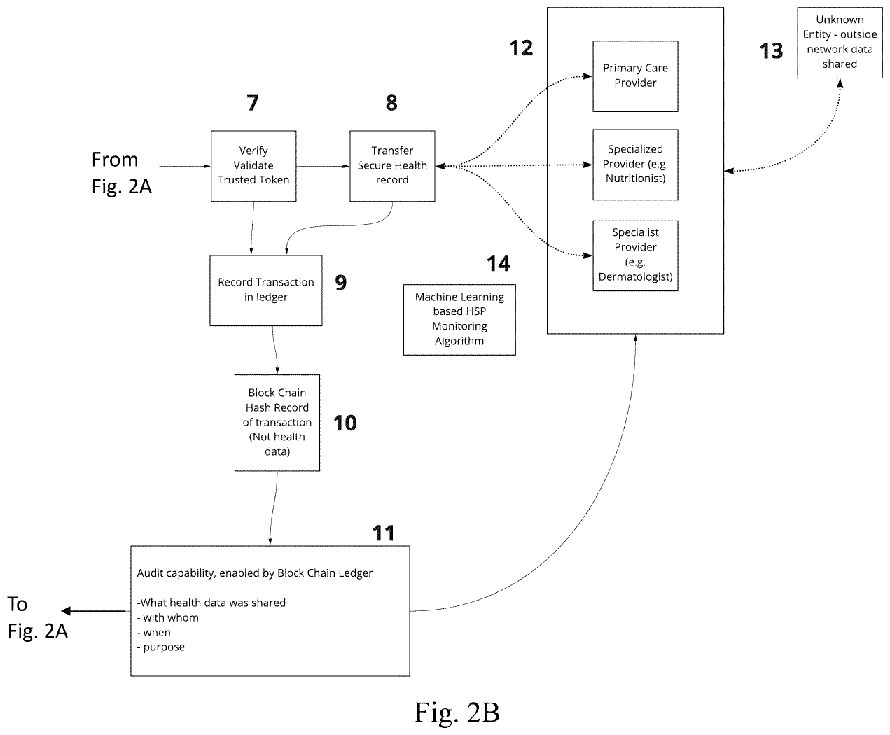

FIGS. 2A-2B illustrate a process of healthcare record transfer and sharing between consumers, different providers, and facilities using a blockchain-based approach. In various embodiments, blockchain-based implementation provides for a secure, managed, certifiable, and consumer-controlled process of transferring and/or sharing healthcare records between two parties (e.g., an electronic health record server and a healthcare provider). In various embodiments, the blockchain-based system acts as a secure service bus for healthcare related information under control of the consumer.

In various embodiments, a consumer 1 may be a member of a HSP system. In various embodiments, the consumer 1 may opt in to the blockchain-based healthcare record transfer system. In various embodiments, the consumer 1 may be included in the HSP because the insuring entity is providing the health service as part of the health plan of the insured. In various embodiments, the consumer 1 may be able to control and/or audit the distribution of their healthcare data (e.g., written medical records, medical imaging, reports, studies, etc.) to the selected HSP network providers. In various embodiments, the consumer may include, exclude, and/or remove providers from receiving the healthcare records and/or any updates to the healthcare records. In various embodiments, the consumer may be any individual whose health record is being protected and/or tracked.

In various embodiments, the consumer 1 may install a healthcare records tracking application at their computer (e.g., personal computer, laptop, etc.) or mobile device (e.g., cell phone, tablet, etc.). In various embodiments, the consumer 1 may interact with the blockchain-based healthcare record-tracking platform via a mobile or web application. In various embodiments, the application may be able to be run on commercially-available mobile and PC hardware. In various embodiments, microservices can be provided for integration into 3rd-party applications and devices. In various embodiments, the application may provide: secure login to the application, viewing and selection of the target provider to send the healthcare record to (provider may be a member of the secure HSP network), the selection of a subset of the health record to deliver to the target provider, and review and submission of the transaction for delivery to the provider. In various embodiments, the transaction in which authorized data is provided to a healthcare provider is a transfer and/or share of the protected healthcare data. In various embodiments, a transfer may include copying the data from a healthcare record database (e.g., EHR) and delivering the identical copy to the selected provider. In various embodiments, sharing may include temporarily providing access to data from a healthcare record database. In various embodiments, the shared data becomes unavailable (e.g., inaccessible) at a predetermined later time. In various embodiments, the HSP provides the consumer the ability to view a history of confirmed transactions that were made sharing the health records with selected providers. In various embodiments, the consumer may select other options, including but not limited to: the selected provider continuing to receive updates of the consumer's healthcare records and the ability for the provider to provide updates to the consumer's health record. In various embodiments, the HSP system may send and/or receive FHIR-based data transactions, using known protocols for delivering healthcare record transactions. In various embodiments, the HSP system may display a digital code (e.g., custom generated QR code) on a mobile device that may be shared with the provider to initiate a transfer of health record transaction. In various embodiments, the code may be a unique, one-time use code generated by the HSP system and configured to be presented on the mobile device, read by the provider (e.g., a provider mobile device), and then sent back to the HSP system by the HSP provider application to the HSP system and validated.

In various embodiments, one or more secure provider 3 may be selected from a database of enrolled and/or approved providers within the HSP system. In various embodiments, only providers enrolled in the HSP system may be eligible for certified tracking and delivery of the consumer's health record. In various embodiments, this list of enrolled secure providers 3 may be continuously updated with new providers as they sign on to the HSP system. In various embodiments, the HSP system may have user-controlled permissions, including but not limited to: which secure providers can and cannot request or receive records/updates, which secure providers can update the health records, and/or the blocking of secure providers from any and all communications with the user.

In various embodiments, the consumer may select portions (e.g., all) of the healthcare record information 4 that are to be sent to the provider via the HSP system. In various embodiments, the user may select all of the available healthcare record. In various embodiments, the user may select a subset of the available healthcare record. In various embodiments, the portions of the healthcare information may be organized into categories. In various embodiments, the categories may be hierarchically organized. In various embodiments, the categories may include supersets of information within the healthcare record. In various embodiments, the categories may include subsets (e.g., finer details) of information within the healthcare record. For example, a consumer may only want certain record details sent to their nutritionist (e.g., blood pressure, age, weight) and so the HSP system module provides only the selected healthcare record details to a packaging/formatting software module to ultimately be sent to the selected healthcare provider.

In various embodiments, the selectable options for portions of the healthcare record may be dynamic based on populated sections of the healthcare record. In various embodiments, a recommended subset may be provided to the consumer. In various embodiments, the recommended subset may be determined using a rules-based algorithm. In various embodiments, the recommended subset may be determined via collaborative filtering. In various embodiments, the recommended subset of the user's healthcare record may be determined based on the services being selected. For example, if the consumer was sending a record to a chiropractor, skeletal and muscular information may be recommended for the transmission to the chiropractor in addition to the basic set of healthcare record. In another example, if a consumer is sending healthcare data to a cardiac care provider, blood work, any heart-related lab work, and/or prescriptions may be recommended for transmission to the cardiac care provider. In various embodiments, the system may recommend a subset of the health record based on machine learning. In various embodiments, the system may recommend a subset of the health record based on sharing patterns across the HSP network.

In various embodiments, healthcare records of a user are stored at a certified datastore 5 (e.g., BCBS Blue) or certified EHR storage partners, including profile record storage. In various embodiments, this allows a HSP to have a universal master data identifier for a user and link the user across different providers to connect the user's EHR Records. In various embodiments, a sample profile might include: Name, HSP ID, Address, Mobile number, Email address, Insurance IDs, Pointers to users records of other systems IDs (if needed and/or desired by user). In various embodiments, the user profile (e.g., insurance member record) may include user profile information generally needed to fulfill the profile of potential receiving systems in view of any healthcare record transfer standards.

In various embodiments, records from the healthcare records may be available via any suitable EHR/EMR database platform. In various embodiments, the EHR platform may be centralized at a single location (e.g., at a hospital). In various embodiments, the EHR platform may be distributed. In various embodiments, the healthcare records may be sent and received securely using known security protocols for transmitting sensitive healthcare data. In various embodiments, healthcare records may be updated securely via the system (e.g., if the consumer enables the provider for update capability.) In various embodiments, updating of healthcare records may be performed as an encrypted HL7 transaction. In various embodiments, other profile information details may be connected to a user's profile via third party sources. For example, a user may link an account associated with a fitness tracking device such that the user's healthcare record is updated in real-time with fitness data. In various embodiments, profile information details may include demographic information (e.g., location of work or home, local air and/or water quality, other relevant information) desired to be packaged and sent via the system. In various embodiments, healthcare record details may be aggregated and formatted into a standard package 6 that can be received by any suitable healthcare system or transfer protocol (e.g., FHIR). In various embodiments, sending the profile information includes selecting the desired profile items, formatting into a data format compatible with the receiving system (leveraging a standard protocol), encrypting and transmitting with a standard interface.

In various embodiments, packaging of healthcare data may include sourcing data records from one or more source systems. In various embodiments, packaging of healthcare data may include assembling the data (from different sources) with a predetermined format of data transfer for the requesting system and/or protocol. For example, a FHIR-based record exchange between systems has a predetermined format that allows the systems to process details of the healthcare records such that they can be properly assembled and placed in the HL7 systems data fields. In various embodiments, predetermined formats may include structured data. In various embodiments, predetermined formats may include unstructured data. In various embodiments, unstructured data may be appropriately tagged as to allow a requesting system to parse the data while following HSP network audit and tracking requirements.

In various embodiments, a secure provider may be verified before a user is permitted to select the secure provider from a list of secure providers to which a healthcare record is to be sent. In various embodiments, the verification process may include various checks and approvals based on data points and/or validation signals of an institute or individual provider (e.g., Hospital, HealthCare organization—Payer, Pharmacy, Non-Healthcare Entities). In various embodiments, verification processes may be performed using one or more APIs. In various embodiments, verification processes may be performed using one or more File Feeds. In various embodiments, verification processes may be performed manually (e.g., through an automated calling or email system). In various embodiments, a verification process may be performed in partnership with key credentialing systems/agencies. In various embodiments, the credentialing system may include a government or non-government agency (e.g., DEA, ADMS, OIG, NCQA, and/or other CVO service providers, such as, Aperture, HCAS, etc.) In various embodiments, the verification process may include analyzing industry standard data points and/or identifications (e.g., CAQH ID, National Provider Identifier, License Number, DEA Number, etc.).

In various embodiments, data points used for verification may include: a Dunn and Brad Street Number and/or mailing address, company formation details, background check information, credit check information, bank account information, etc. In various embodiments, a verification process may include: confirming that the secure provider is verified and legitimate, validating that the secure provider is authorized to request/receive given data. In various embodiments, at the time of joining the HSP network, the details of the secure provider may be stored in a provider profile at a database within the HSP network. In various embodiments, as part of a HSP subscription agreement, the secure provider may sign various authorizations to request/receive data via the HSP network and its participant entities (e.g., Payers, Providers, Pharmacies, Employers and Individual Users). In various embodiments, Providers may also be assigned various system identifiers and/or secure tokens for use in securing any transactions within the HSP network. In various embodiments, a commercially-available verification process may be used, such as the services from RISKCONNECT. In various embodiments, a verification process may assess each vendor/provider according to various industry standards, create an audit trail of documentation, send alerts when verification documents are expiring, communicate third-party risk with dashboards and point-and-click reports, etc.

In various embodiments, a healthcare record may be securely transferred 8 from a healthcare record database to one or more selected secure providers via any suitable secure communications protocol for transferring healthcare records (e.g., FHIR). In various embodiments, the secure provider verification process may merge the secure provider's information (e.g., name of provider, address, medical license number, token, etc.) with the health record information and securely communicate with the provider system application to transfer the health record information. In various embodiments, the merging of the secure provider information into the healthcare record information will be performed in sync with the creation of the transaction ledger of the record of transfer. The transmission of the health record would be enabled using standard FHIR (formats and secure protocols). In various embodiments, hashing is performed after the data is merged into the package.

In various embodiments, the secure provider may be assigned a token as a unique identifier (ID). In various embodiments, the unique ID may be stored in a HSP provider datastore. In various embodiments, the HSP provider datastore may include a list of verified providers, provider information (e.g., name, address, medical license number, etc.), and the unique ID. In various embodiments, the datastore may include a relational database (e.g., SQL). In various embodiments, the datastore may include a non-relational database (e.g., MongoDB) In various embodiments, the unique ID and be leveraged in the recorded transaction on chain data as the who with transaction ID. In various embodiments, the HSP network may use at least two (2) different tokens for the secure providers. In various embodiments, the HSP network may use one or more additional secure tokens associated with the consumer(s) for hashing. In various embodiments, one or more token(s) associated with the provider may be included in the medical document package. In various embodiments, any tokens associated with the consumer and/or the providers may be included in the recorded blockchain record.

In various embodiments, the healthcare record transfer 8 may be a PUSH or PULL transaction. In various embodiments, the one or more selected secure providers may be notified that a user's (e.g., consumer's) record is ready to secure transfer. In various embodiments, the healthcare record transfer 8 can be requested (e.g., scheduled) by each secure provider at a time that is convenient for them. In various embodiments, a healthcare record may be PUSHED automatically into a secure provider's system if it is configured to do so. In various embodiments, a provider's system may accept an API call from the HSP system. In various embodiments, the API call may be configured as a notification indicating the provider should make a call to the HSP to retrieve the record. In various embodiments, the secure provider's system may be configured to directly receive an API call to receive and process the incoming record automatically.

In various embodiments, the transaction (i.e., transfer of healthcare record) may be recorded in a blockchain ledger 9. In various embodiments, once the transaction is verified and accepted as true by the entire network, the transaction may be recorded to the blockchain ledger 9. In various embodiments, the steps for recording transactions to blockchain may include: transfer of the health record; if transfer was successful, then the transfer is recorded on the blockchain ledger; details of transaction (e.g., receiver, member, date time, hash of transaction) may be recorded; audit details of transactions and hash of transaction are recorded in audit log; a block chain record is created; a hash of the blockchain record is generated; and the blockchain is stored and shared on a network (e.g., P2P) ledger.

In various embodiments, the HSP network operates at least a portion (e.g., all) of the nodes of the blockchain. In various embodiments, the HSP network includes one or more redundant nodes. In various embodiments, each node may include a copy of either the entire ledger or relevant portions of the ledgers. In various embodiments, the HSP network may include one or more (e.g., a plurality of) validator nodes configured to validate the blockchain transactions prior to writing to the ledger. In various embodiments, the HSP network may operate without compensation to the individual nodes (e.g., the nodes are maintained and operated by members of the NSP network). In various embodiments, the HSP system may maintain a separate audit log of every transaction. In various embodiments, the transaction may be recorded on the blockchain ledger 9, which is a private blockchain network. In various embodiments, the audit log also has additional information on the actual record transfers between the parties using FHIR protocol.

In various embodiments, the details of the health record transaction are published on the blockchain ledger. In various embodiments, the details may include the sender information, the recipient information, a hash of the data stored within the healthcare record package, and/or a previous hash. In various embodiments, each block may include one or more of: Transaction ID; Transaction status (e.g., sent, received); content list (e.g., what data items were sent, but not the items themselves); date-time stamps of data; date-time stamps of the transaction; user/consumer identifier; provider identifier; and/or a hash of the healthcare record. In various embodiments, the transaction details may be made available on a private blockchain for providers and consumers to access (e.g., for tracking). In various embodiments, tracking a private blockchain may allow the certification of the history of the HSP transactions made and allow for identification of any potential misuse of consumer information. In various embodiments, the private blockchain may include a minimum of seven nodes. In various embodiments, the private blockchain may require 66% of nodes agreeing for consensus. In various embodiments, a commercially-available blockchain may be utilized, such as Amazon Hyperledger.

In various embodiments, a 10 hash may be recorded in the blockchain ledger that does not include any healthcare information. In various embodiments, the healthcare record that is transferred may be hashed. In various embodiments, any transaction IDs may be hashed along with any tokens.

In various embodiments, consumers and/or secure providers may be provided access (e.g., via an application interface) to the blockchain records. In various embodiments, the consumer and/or secure providers can view the details of any past transactions of healthcare information to which they are a party (e.g., sender or recipient). In various embodiments, a login may be provided to each of the sender (e.g., the consumer) and the recipient (e.g., the secure provider). In various embodiments, an audit report can be provided on demand. For example, the HSP system may receive a request from a user (e.g., consumer or provider) to audit the blockchain. In various embodiments, the blockchain ledger may be queried based on the received audit request. In various embodiments, the internal HSP transaction log, individual member record, and/or secure provider records may be cross-verified against the blockchain ledger. In various embodiments, each blockchain transaction may include a reference ID. For example, a stored hash of the transaction in the blockchain can be validated against a stored hash in the HSP audit log, which can also be validated against the transaction information in the log. In various embodiments, the internal HSP transaction log can be validated against the blockchain ledger as needed.

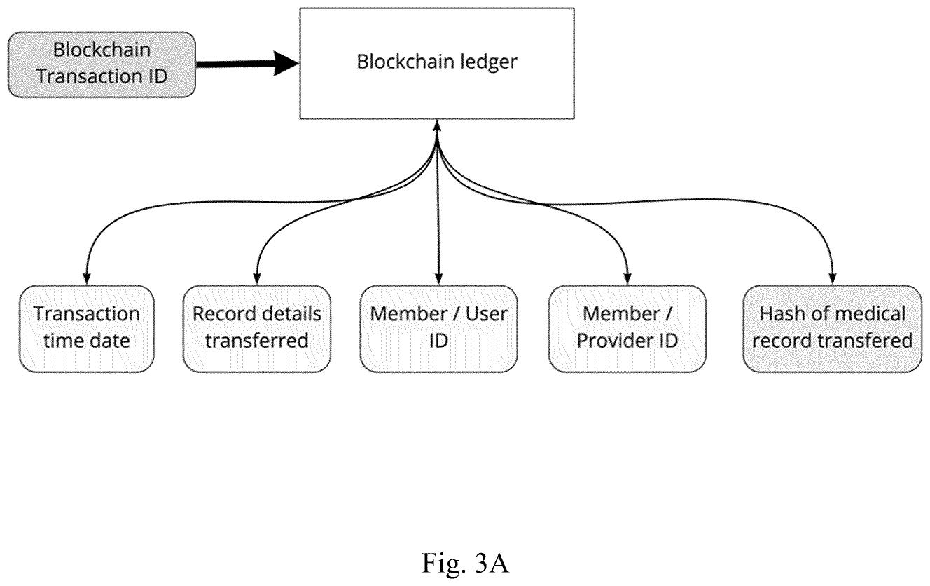

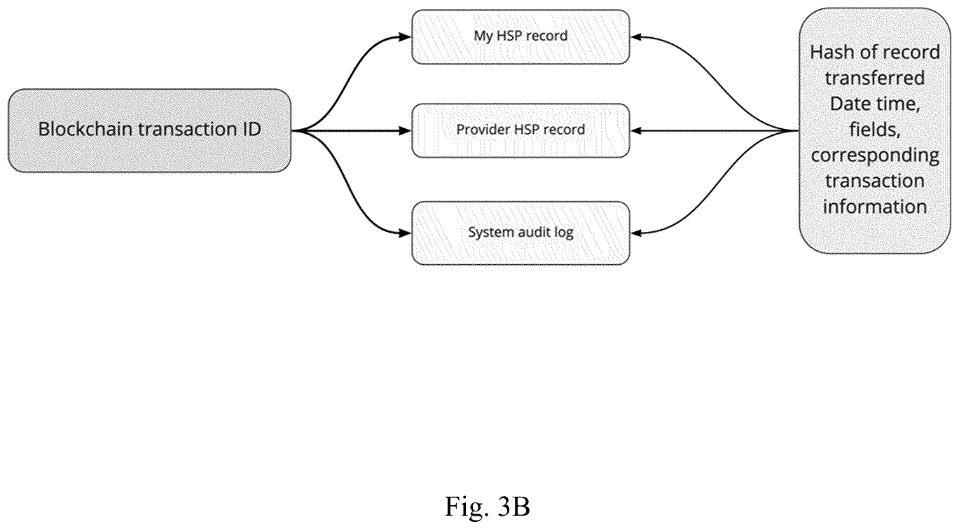

FIG. 3A illustrates a flow diagram of a process for recording a transaction on a blockchain ledger. As shown in FIG. 3A, a transaction time and date, details of the records transferred, member/user ID, member/provider ID, and/or a hash of the medical record data transferred may be stored in a blockchain ledger. In various embodiments, each entry in the blockchain ledger includes a blockchain transaction ID. FIG. 3B illustrates a flow diagram of a process for storing a transaction ID and hash in an HSP system. In various embodiments, the blockchain transaction ID may be provided to one or more of: a consumer's HSP record, a provider's HSP record, and/or a system audit log. In various embodiments, the hash of the medical record data transferred, transaction time and date, fields within the record, and/or corresponding transaction information may be provided to one or more of: a consumer's HSP record, a provider's HSP record, and/or a system audit log.

In various embodiments, various details of a health care record transaction within the HSP may be recorded and stored in a system audit log. In various embodiments, the HSP system audit log may record transaction details (e.g., hash, actual health data, the parties involved in the transaction, date/timestamp, blockchain ID, etc.). In various embodiments, the system audit logs may be used to determine what information was sent, when the information was sent, to whom, and from whom. In various embodiments, the system audit logs may be used in conjunction with the hash and blockchain ID to audit a transaction.

In various embodiments, the audit capability can be provided with a specific HSP applications installed on a computing device. In various embodiments, the audit capability can be provided via one or more APIs configured to interface with third-party systems. In various embodiments, all transactions related to member data may be recorded in the HSP blockchain ledger. In various embodiments, the HSP blockchain ledger may be used to create a transactional log and enabling access to providers and consumers to certify/validate and audit historical transactions. In various embodiments, the HSP system may include an internal transactional log and blockchain ledger. In various embodiments, healthcare record data changes may be recorded in an audit log for the healthcare record database. In various embodiments, any APIs may utilize known communications protocols for transmitting healthcare information, such as, for example, FHIR and HL7.

In various embodiments, data stored in the blockchain ledger may include: Date-time records prepared, healthcare record segment fields, secure provider, date-time transaction successfully executed, requestor, transaction type (new, update, etc.), data aggregating sources, etc. In various embodiments, the transaction ledger is a single blockchain ledger. In various embodiments, other audit databases may be queried when conducting an audit of the HSP system transactions. For example, the other audit databases may include: profile store (members and providers), log of changes, system orchestration store and consumer or provider activity, health record store and change log of record changes, LDAP store and trail of changes, system admin access, and/or key management activities.

In various embodiments, an exemplary audit workflow for the recording of a transaction and sending of data to a provider includes a request from a user that consists of one or more secure provider and specific healthcare record details (e.g., portions of the health record) to be delivered. In various embodiments, the HSP system may prepare the data from a healthcare record datastore. In various embodiments, the HSP system may request healthcare record information from other sources that may format, validate, and/or transmit the healthcare record information to the HSP providers system. In various embodiments, the HSP providers system may respond with execution acknowledgment. In various embodiments, the blockchain ledger may be updated with the record of the transaction.

In various embodiments, the user (e.g., a consumer) may provide a request for a transaction log of all health record activity and/or history. In various embodiments, the transaction log may be provided including all HSP transactions related to a user's health information (e.g., all instances where the user sent their health information to a secure provider). In various embodiments, the transaction log may allow the user to view a list of transactions with information related to when, with whom, and what healthcare record information was shared or transferred within the HSP system.

Yes, audit reports could be requested by consumer or providers related to their transactions.

### EXAMPLE

As an example, a user may request a report of all health record data delivered between date A and B and may specify a secure provider. In various embodiments, a user may request a report of all secure providers who received healthcare records between those two dates. In another example, a secure provider may request a report of all healthcare record data received between data A and B for a specific user X. In various embodiments, the secure provider may request a report of all healthcare records received between those two dates.

In various embodiments, secure providers in the HSP system may be contractually bound to abide by HSP rules. In various embodiments, the secure providers may not be allowed to share health records with other providers or other entities outside the HSP system or share data without processing it through the ecosystem. In various embodiments, a member/consumer of the HSP network may submit a request to the HSP to scan a HSP provider/pharmacy/entity for any existing health records for that member/consumer. In various embodiments, the scan may automatically occur when a new HSP provider/pharmacy/entity is onboarded to the HSP network. In various embodiments, if an existing HSP health record is found with HSP provider/pharmacy/entity, the member/consumer may be notified. In various embodiments, the HSP network may search the system audit logs on behalf of a member/consumer to determine which existing HSP provider(s) is/are storing their healthcare record information. In various embodiments, the HSP network may search the system audit logs to determine if a non-HSP provider/pharmacy/entity was sent healthcare record information.

In various embodiments, secure providers may be vetted approved and integrated into the HSP system and secured with appropriate cryptographic based identification. In various embodiments, each provider may be identified with a unique ID (e.g., a token) and the HSP system would generate unique token(s) generated by the HSP HSM based function that would be leveraged to secure transactions with the specific provider. These tokens will be unique and can be rolled in case of a breach in token security.

In various embodiments, if secure providers are enabled and/or allowed for bidirectional exchange including record update, provider transactions will become part of the HSP system record and, thus, be recorded as a new block in the blockchain.

In various embodiments, the provider may interact with the HSP platform in a manner that they would be able to receive the members health record appropriate to the specific provider. In various embodiments, an application configured for the secure provider may allow for, among other things: secure login to the HSP system, a dashboard, and/or listing of information shown for health record information to be transferred. In various embodiments, a provider may be allowed to select one or more records to be transferred. In various embodiments, the one or more records may be transferred in the form required by a system and/or by law (e.g., FHIR). In various embodiments, when the provider transacts (e.g., transfers or shares) healthcare records, the transaction may be performed as a secure transactional exchange with the HSP system such that an audit record can be created by the HSP indicating that the health record transfer has occurred, when and what was transferred.

In various embodiments, the provider application may include a code scanning module configured to initiate a pull request of a particular user's health record. In various embodiments, the code scanning module may be configured to scan a unique digital code (e.g., QR code). In various embodiments, a unique, one-time code may be generated by the HSP. In various embodiments, the code may be generated on-demand by, for example, activating a button in the consumer or provider application. In various embodiments, the one-time code may be sent to user's mobile device to be read by the providers system. In various embodiments, other known methods of initiating a pull request may be used, including, for example, optical, Bluetooth, NFC, and/or RFID. In various embodiments, once the code is read by the provider's application, the pull request is sent to the HSP system to be validated, and the transaction released if the provider is validated as a secured provider. In various embodiments, the user's health record may be automatically updated. In various embodiments, the user's health record may be manually updated. In various embodiments, the provider may send an update to the HSP system for adding to, updating and/or refreshing the user's health record. In various embodiments, any updates, additions, and/or refreshes may be recorded in a separate block on the blockchain. In various embodiments, only the data that changed may be hashed and stored in a new block on the blockchain. In various embodiments, if the healthcare record contents (e.g., new exam or study added, new clinical notes added, data removed, etc.) have changed, the entire record contents may be rehashed.

In various embodiments, the record transfer to the provider could be either PUSH or PULL depending on the transactional need and the integration aspects of the providers system. In various embodiments, processing of a request may be initiated in a number of ways. For example, a secure provider may view a queue of records awaiting delivery in the HSP application, select one or more records awaiting transfer and PULL the record(s). In another example, the secure provider may have an integration with their record system (e.g., EHR/EMR) that automatically retrieves and integrates the healthcare record into their record system, and acknowledges the record intake.

In various embodiments, the HSP system may provide a set of secure APIs/microservices that would allow for an integration with a provider's system for direct system to system connection with the providers patient EMR platform (e.g., EPIC or other FHIR compatible system). In various embodiments, microservices and/or integrations may be provided for other smart devices.

In various embodiments, alerts and/or notifications may be provided in the event a provider shares a healthcare record with an entity outside of the HSP network. In various embodiments, transactions outside the HSP network may be flagged (e.g., through one or more alerts) to the consumer when an in-network HSP participant attempts to share data or executes a data share with a non-HSP certified entity. In various embodiments, a user may set a permission that a secure provider is permitted to share their healthcare record(s) with out-of-network entities. Where a user has set permissions for out-of-network transfer, no alerts may be raised. In various embodiments, when a HSP member tries to share data outside the HSP network with a non-HSP entity, a flag may be thrown by the HSP system, and the user may be notified that an unauthorized share with an out-of-network entity occurred. In various embodiments, the HSP system may warn the provider if the provider is about to share user healthcare record(s) with an out-of-network entity. In various embodiments, alerts may be delivered via standard digital channels like SMS text messaging, e-mails, application notifications, etc. In various embodiments, non-HSP entities may be tracked by comparing the entity to the registered HSP entities to determine if the entity isn't a member to the HSP network. If the entity is determined to not be a member to the HSP network, then that entity may be marked NON-HSP entity. In various embodiments, entity information may be checked against a HSP entity database of HSP subscribed vs. non-subscribed entities. In various embodiments, HSP non-subscribed entities may be added from 3rd party data sources and marked as non-HSP entities if they are not found in the existing HSP subscribers list. In various embodiments, each transaction between a HSP-subscribed entity and non-subscribed entity may be recorded in the blockchain ledger and/or the audit logs.

In various embodiments, the system may include a HSP monitoring module 14. In various embodiments, the HSP monitoring module 14 may include a machine learning algorithm. In various embodiments, the HSP monitoring module 14 may include a neural-network. In various embodiments, the HSP monitoring module 14 may learn and predict any variances of data sharing. In various embodiments, the HSP monitoring module 14 may provide an alert to HSP admins and/or consumers when abnormal data sharing is detected. In various embodiments, a machine learning algorithm may be trained on HSP entity databases, transaction system logs, and/or other suitable 3rd party data. In various embodiments, logs may contain data, such as, for example, users sharing certain health records and sending to HSP entities or other transactions. In various embodiments, as the logs grow, the machine learning algorithm may be re-trained on the updated data. In various embodiments, the machine learning algorithm may continue to analyze and look for patterns as the system audit log and/or the blockchain ledger changes. In one example, the machine learning algorithm may recognize a pattern that a provider of type A has been sent data that is typically not needed or sent to that provider. In that example, the machine learning algorithm may flag this unusual transaction to the member/consumer. In another example, the machine learning algorithm may recognize an unusual transaction where a user's data simultaneously being requested to be shared with different entities in physically separate locations by a user at a different geographical location themselves. In various embodiments, the machine learning algorithm may include a neural network. In various embodiments, the neural network may include a recurrent neural network. In various embodiments, the machine learning algorithm may include at least one of: Random Forest, AdaBoost, Gaussian Mixture Modelling (GMM), and/or k-means clustering.

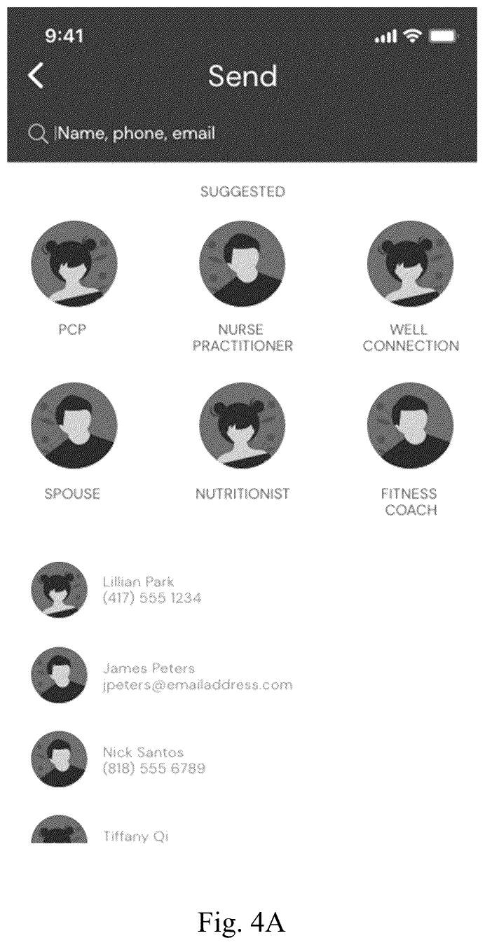

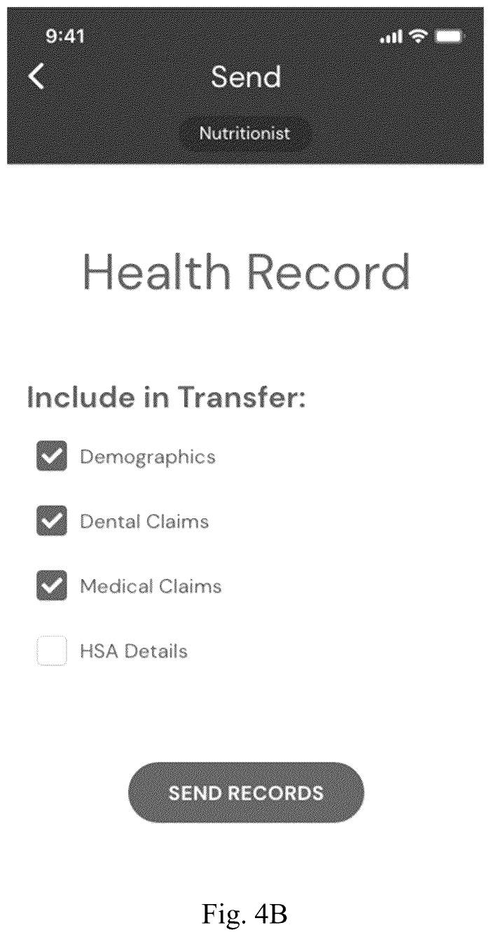

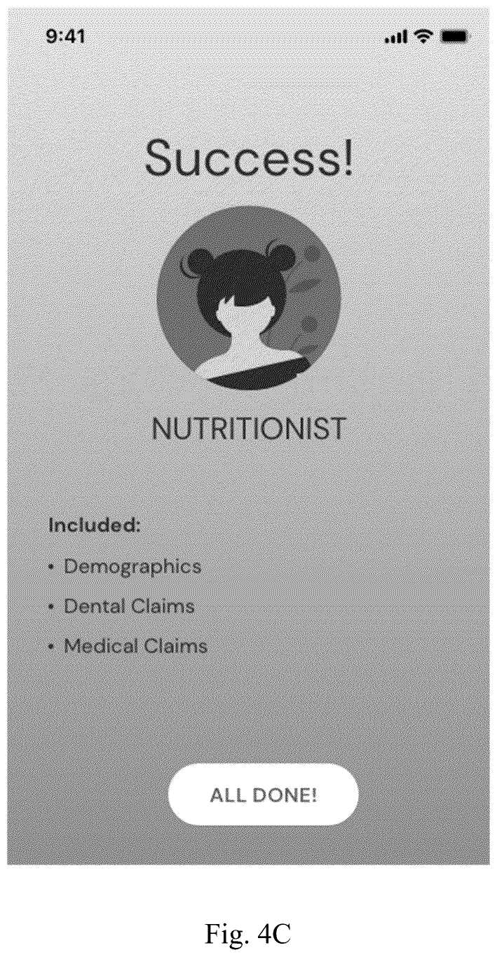

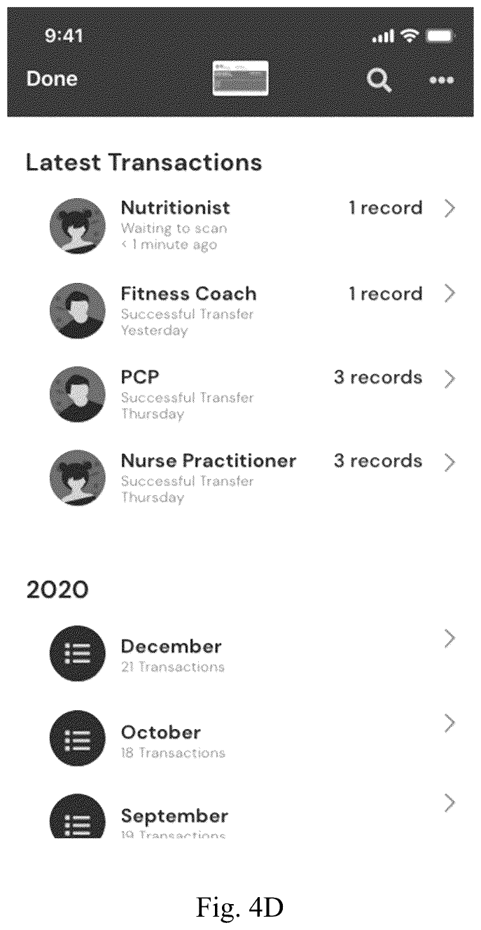

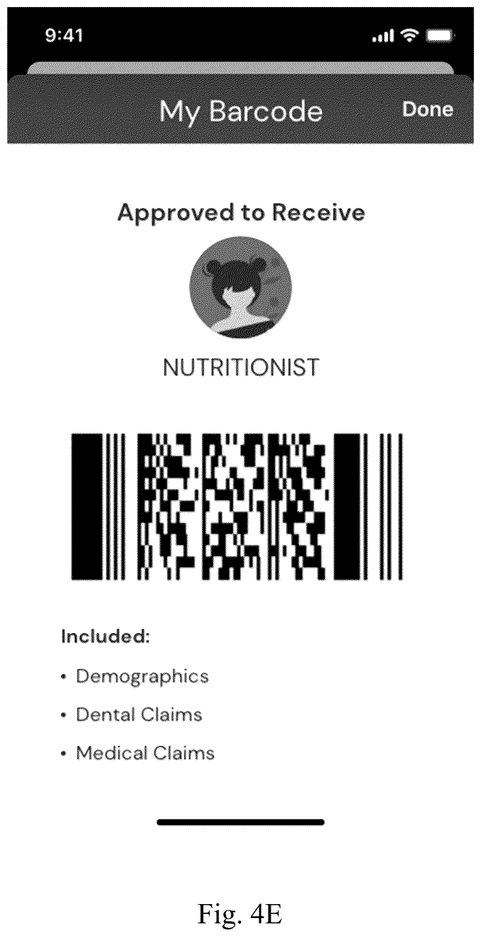

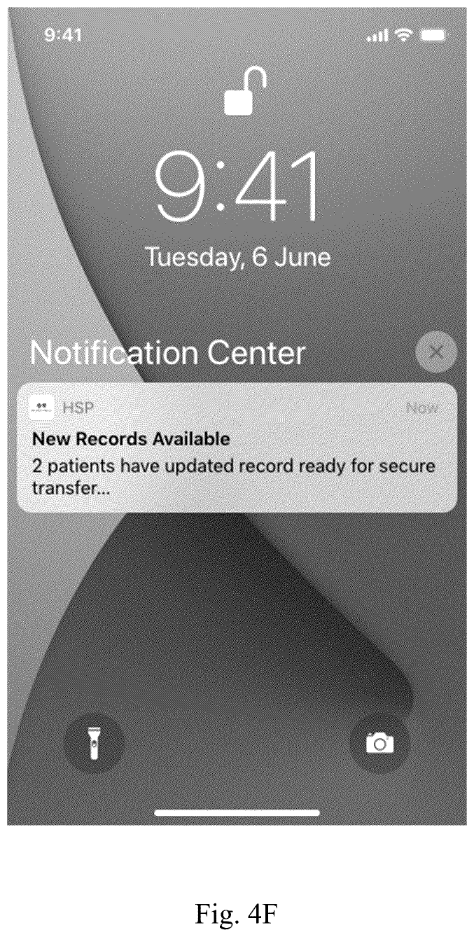

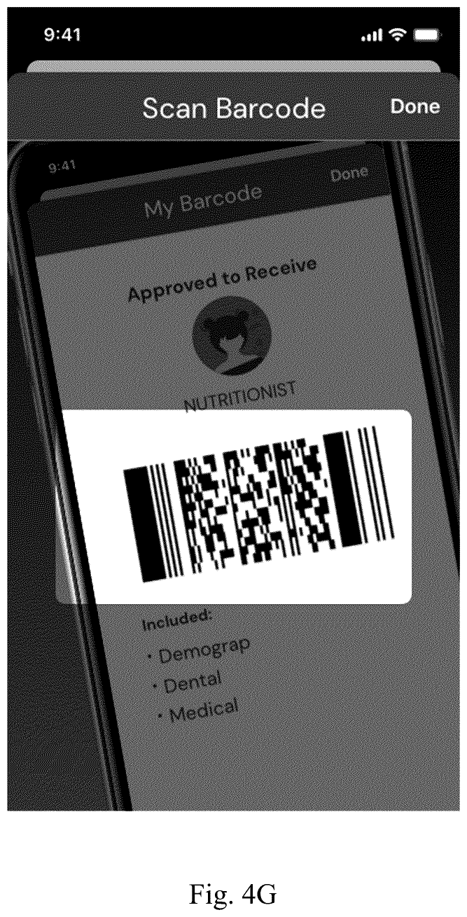

FIGS. 4A-4G illustrate various snapshots of a mobile phone application. FIG. 4A illustrates an exemplary provider selection page displayed on a mobile device where a user may select one or more providers to which a selected set of healthcare records will be sent. For example, a user may select a nutritionist to send one or more healthcare records. FIG. 4B illustrates an exemplary record selection page displayed on a mobile device where a user may select and/or deselect components of a medical record to send to a specific provider. For example, a user may select “demographics,” “dental claims,” and medical claims” to be securely sent to a nutritionist. In various embodiments, all options may be selected by default and the user may deselect one or more options, for example, the “HSA details.” FIG. 4C illustrates a page displayed upon the successful transfer of a healthcare record to a specific provider (e.g., a nutritionist). In various embodiments, the success page may display a summary of the information transferred to the specific provider (e.g., demographics, dental claims, and medical claims). FIG. 4D illustrates an exemplary page listing the latest transactions (e.g., healthcare record shares/transfers) initiated by the user for each time period (e.g., year). FIG. 4E illustrates an exemplary barcode page displaying a bar code generated by the application that is configured to initiate a transfer of one or more healthcare records to a specified healthcare provider. FIG. 4F illustrates an exemplary lock screen on a mobile device having a notification to a provider that new healthcare records are available for secure transfer. FIG. 4G illustrates an exemplary scanning function of an application configured to scan a barcode, such as the barcode illustrated in FIG. 4E.

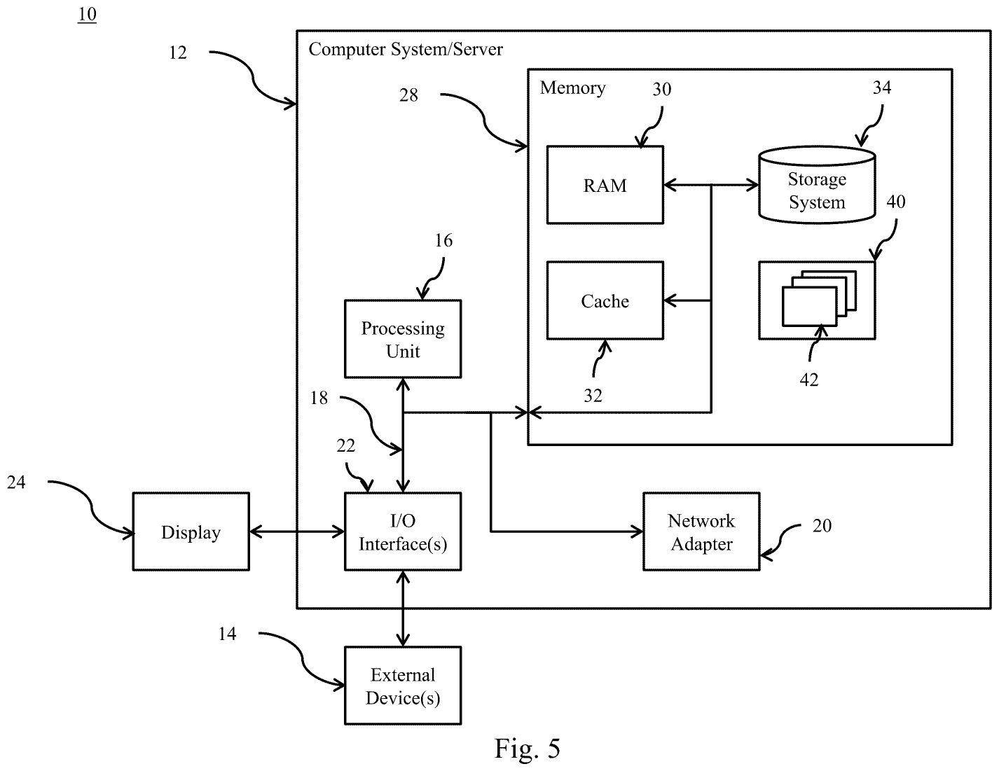

Referring now to FIG. 5, a schematic of an example of a computing node is shown. Computing node 10 is only one example of a suitable computing node and is not intended to suggest any limitation as to the scope of use or functionality of embodiments of the invention described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 5, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

A Picture Archiving and Communication System (PACS) is a medical imaging system that provides storage and access to images from multiple modalities. In many healthcare environments, electronic images and reports are transmitted digitally via PACS, thus eliminating the need to manually file, retrieve, or transport film jackets. A standard format for PACS image storage and transfer is DICOM (Digital Imaging and Communications in Medicine). Non-image data, such as scanned documents, may be incorporated using various standard formats such as PDF (Portable Document Format) encapsulated in DICOM.

An electronic health record (EHR), or electronic medical record (EMR), may refer to the systematized collection of patient and population electronically-stored health information in a digital format. These records can be shared across different health care settings and may extend beyond the information available in a PACS discussed above. Records may be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

EHR systems may be designed to store data and capture the state of a patient across time. In this way, the need to track down a patient's previous paper medical records is eliminated. In addition, an EHR system may assist in ensuring that data is accurate and legible. It may reduce risk of data replication as the data is centralized. Due to the digital information being searchable, EMRs may be more effective when extracting medical data for the examination of possible trends and long term changes in a patient. Population-based studies of medical records may also be facilitated by the widespread adoption of EHRs and EMRs.

Health Level-7 or HL7 refers to a set of international standards for transfer of clinical and administrative data between software applications used by various healthcare providers. These standards focus on the application layer, which is layer 7 in the OSI model. Hospitals and other healthcare provider organizations may have many different computer systems used for everything from billing records to patient tracking. Ideally, all of these systems may communicate with each other when they receive new information or when they wish to retrieve information, but adoption of such approaches is not widespread. These data standards are meant to allow healthcare organizations to easily share clinical information. This ability to exchange information may help to minimize variability in medical care and the tendency for medical care to be geographically isolated.

In various systems, connections between a PACS, Electronic Medical Record (EMR), Hospital Information System (HIS), Radiology Information System (RIS), or report repository are provided. In this way, records and reports form the EMR may be ingested for analysis. For example, in addition to ingesting and storing HL7 orders and results messages, ADT messages may be used, or an EMR, RIS, or report repository may be queried directly via product specific mechanisms. Such mechanisms include Fast Health Interoperability Resources (FHIR) for relevant clinical information. Clinical data may also be obtained via receipt of various HL7 CDA documents such as a Continuity of Care Document (CCD). Various additional proprietary or site-customized query methods may also be employed in addition to the standard methods.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
receiving a request from a user for one or more healthcare records;
receiving a selection of one or more secure providers from the user;
retrieving the one or more healthcare records from a healthcare record database;
combining the one or more healthcare records into a healthcare record package;
generating a hash based on the healthcare record package;
transmitting the healthcare record package to the one or more secure providers; and
recording the user, the one or more secure providers, and the hash at a block in a blockchain ledger.

2. The method of claim 1, wherein the medical document package comprises a token associated with the user.

3. The method of claim 2, wherein the medical document package comprises one or more additional tokens associated with the selected one or more secure providers.

4. The method of claim 2, wherein each token comprises a unique identifier.

5. The method of claim 1, wherein generating the hash comprises hashing data within the medical document package.

6. The method of claim 1, further comprising generating a transaction log for the recording in a log database.

7. The method of claim 1, further comprising receiving, from the user, an audit request for a transaction recorded on the blockchain.

8. The method of claim 7, further comprising querying the blockchain ledger based on the audit request.

9. The method of claim 8, further comprising providing the hash in response to the audit request.

10. The method of claim 9, further comprising comparing a recorded token to a user token to verify that the user was associated with the transaction.

11. The method of claim 1, wherein the medical record database comprises an electronic health record (EHR) server.

12. The method of claim 1, wherein the block does not include any data from the one or more healthcare records.

13. A system comprising:
a healthcare record database;
a computing node comprising a computer readable storage medium having program instructions embodied therewith, the program instructions executable by a processor of the computing node to cause the processor to perform a method comprising:
receiving a request from a user for one or more healthcare records;
receiving a selection of one or more secure providers from the user;
retrieving the one or more healthcare records from the healthcare record database;
combining the one or more healthcare records into a healthcare record package;
generating a hash based on the healthcare record package;
transmitting the healthcare record package to the one or more secure providers; and
recording the user, the one or more secure providers, and the hash at a block in a blockchain ledger.

14. A computer program product for continuous clinical evaluation and care adjustment, the computer program product comprising a computer readable storage medium having program instructions embodied therewith, the program instructions executable by a processor to cause the processor to perform a method comprising:
receiving a request from a user for one or more healthcare records;
receiving a selection of one or more secure providers from the user;
retrieving the one or more healthcare records from a healthcare record database;
combining the one or more healthcare records into a healthcare record package;
generating a hash based on the healthcare record package;
transmitting the healthcare record package to the one or more secure providers; and
recording the user, the one or more secure providers, and the hash at a block in a blockchain ledger.
